(19) United States
(12) Patent Application Publication
(10) Pub. No.: US 2011/0184447 A1
(43) Pub. Date: Jul. 28, 2011

(54) SURGICAL CUTTING TOOL AND METHOD

(75) Inventors: Carla R. Leibowitz, San Carlos, CA (US); Christopher U. Phan, San Leanadro, CA (US)

(73) Assignee: WARSAW ORTHOPEDIC, INC., Warsaw, IN (US)

(21) Appl. No.: 12/693,717

(22) Filed: Jan. 26, 2010

(51) Int. Cl. A61B 17/32

(52) U.S. Cl. 606/170

(57) ABSTRACT

Embodiments of the invention include surgical cutting tools with expandable blade portions configured to be inserted through a relatively small opening and expanded in place to manipulate tissue. Some embodiments include expandable blades that may be used to one or more of cut, detach, and remove tissue of or associated with skeletal structures such as one or more vertebrae or portions of the spine or vertebrae. Some embodiments may include use in related methods.

### FIELD OF THE INVENTION

The present invention relates generally to the field of surgically manipulating tissue, and more particularly relates to instruments, implants, and methods that may be used to one or more of cut, detach, and remove tissue such as, but not limited to, spinal disc material located between one or more vertebrae or portions of vertebrae and to treat spinal conditions.

### BACKGROUND

It is sometimes useful in surgical procedures to gain access to a surgical site through a relatively small opening and then to manipulate tissue within a larger volume at the surgical site. Spinal disc material is often detached and removed in association with spinal procedures such as discectomy, spinal fusion, and disc replacement. In these and other spinal procedures, disc material has traditionally been detached and removed with grasping, clipping, cutting, and scraping instruments such as rongeurs, curettes, osteotomes, and scrapers. While many of these instruments do include curved portions and some articulating distal portions, they are generally limited in their ability to reach more than relatively small volumes of tissue at a surgical site when operated through a small opening. Instrument effectiveness is further limited where the access channel from outside of a patient's body to the surgical site is not only narrow but is also long. In this circumstance, angulation of instruments inserted through the access channel is significantly restricted.

The success of many surgical procedures may be severely limited if adequate and appropriate tissue is not detached and removed from a surgical site. Spinal fusion is an example of a procedure which may be ineffective if adequate disc material is not removed and if the endplates of vertebrae to be fused are not sufficiently prepared for new bone growth. Spinal fusions, or other procedures, that are accomplished percutaneously are advantageous to patients because such procedures subject patients to less tissue disruption and trauma. However, many percutaneous procedures limit visualization and the ability to effectively detach and remove tissue and limit effective preparation of other tissues.

Improved surgical instruments that enable adequate detaching and removing of appropriate tissues and preparation of tissues to be treated through narrow operating channels are therefore needed. It may be advantageous for some devices to accomplish these tasks in an improved manner by being introduced into a surgical site through a relatively narrow opening and by including one or more portions that expand and effectively detach, capture, remove, and/or otherwise manipulate tissues to be treated.

### SUMMARY

One embodiment of the invention is a surgical cutting tool that includes a tubular shaft with an expandable blade comprising one or more cuts in the tubular shaft. The expandable blade may be near a distal end of the tubular shaft, and the expandable blade may deploy substantially radially from the tubular shaft. Embodiments of the surgical cutting tool may include an actuator member that extends beyond the distal end of the tubular shaft and includes a bearing surface configured to press against the distal end of the tubular shaft when the actuator member is moved relative to the tubular shaft in a first direction to deploy the expandable blade. The bearing surface of some embodiments is not connected to the tubular shaft such that substantially any tensile resistance exists between the tubular shaft and the actuator member when the actuator member is moved relative to the tubular shaft in a second direction that is substantially opposite from the first direction.

Another surgical cutting tool embodiment of the invention includes a tubular shaft with an expandable blade comprising one or more cuts in the tubular shaft, wherein the expandable blade is near a distal end of the tubular shaft, and wherein the expandable blade deploys substantially radially from the tubular shaft with one or more substantially radially extending elements. Each substantially radially extending element may include two substantially opposite end portions that couple with a main body of the tubular shaft, and a central portion between the two substantially opposite end portions that includes elements for cutting tissue. The central portion of some embodiments is offset from at least portions of the two substantially opposite ends. Embodiments of the surgical cutting tool may also include an actuator member configured to apply force to the tubular shaft when the actuator member is moved relative to the tubular shaft in a first direction to deploy the expandable blade.

Yet another embodiment of the invention is a surgical cutting tool having at least a tubular shaft with an expandable blade comprising one or more cuts in the tubular shaft, wherein the expandable blade is near a distal end of the tubular shaft, and wherein the expandable blade deploys substantially radially from the tubular shaft. The surgical cutting tool may include an actuator member that extends beyond the distal end of the tubular shaft and includes a bearing surface configured to press against the distal end of the tubular shaft when the actuator member is moved relative to the tubular shaft in a first direction to deploy the expandable blade. The actuator member may also include a plunger configured to push material through the expandable blade and out of the distal end of the tubular shaft.

### BRIEF DESCRIPTION OF THE DRAWINGS

FIG. 1 is a perspective view of an embodiment of a surgical cutting tool.

FIG. 2 is a perspective view of the embodiment of the surgical cutting tool of FIG. 1 with portions of the surgical cutting tool removed to more clearly show other portions of the surgical cutting tool.

FIG. 3 is a perspective view of the embodiment of the surgical cutting tool of FIG. 1 with portions of the surgical cutting tool removed to more clearly show other portions of the surgical cutting tool.

FIG. 4 is a perspective view of a distal end the embodiment of the surgical cutting tool of FIG. 1 with an expandable blade in an expanded state.

FIG. 5 is a cross-sectional view near the distal end of the surgical cutting tool of FIG. 1.

FIG. 6 is a perspective view of an expandable blade of an embodiment of a surgical cutting tool with the expandable blade in an expanded state.

FIG. 7 is a cross-sectional view through the expandable blade of FIG. 6.

FIG. 8 is a perspective view of the expandable blade of FIG. 6 with the expandable blade in a contracted state.

FIG. 9 is a perspective view of an expandable blade of an embodiment of a surgical cutting tool with the expandable blade in an expanded state.

FIG. 10 is a cross-sectional view through the expandable blade of FIG. 9.

FIG. 11 is an elevation view of a distal end of an actuator member.

FIG. 12A is an elevation view of the embodiment of the surgical cutting tool of FIG. 1 with portions of the surgical cutting tool removed to more clearly show other portions of the surgical cutting tool, and in an expanded state.

FIG. 12B is an elevation view of the embodiment of the surgical cutting tool of FIG. 1 with portions of the surgical cutting tool removed to more clearly show other portions of the surgical cutting tool, and in a contracted state.

FIG. 12C is an elevation view of the embodiment of the surgical cutting tool of FIG. 1 with portions of the surgical cutting tool removed to more clearly show other portions of the surgical cutting tool, and in a contracted state with a portion of the actuator member extending from the expandable blade.

FIG. 13A is a cross-sectional view of the distal end of the surgical cutting tool in the vertical plane of the elevation view of FIG. 12A.

FIG. 13B is a cross-sectional view of the distal end of the surgical cutting tool in the vertical plane of the elevation view of FIG. 12B.

FIG. 13C is a cross-sectional view of the distal end of the surgical cutting tool in the vertical plane of the elevation view of FIG. 12C.

FIG. 14 is a perspective view of an expandable blade of an embodiment of a surgical cutting tool with the expandable blade in an expanded state.

FIG. 15 is a perspective view of a bristle embodiment of the expandable blade of FIG. 14.

### DETAILED DESCRIPTION

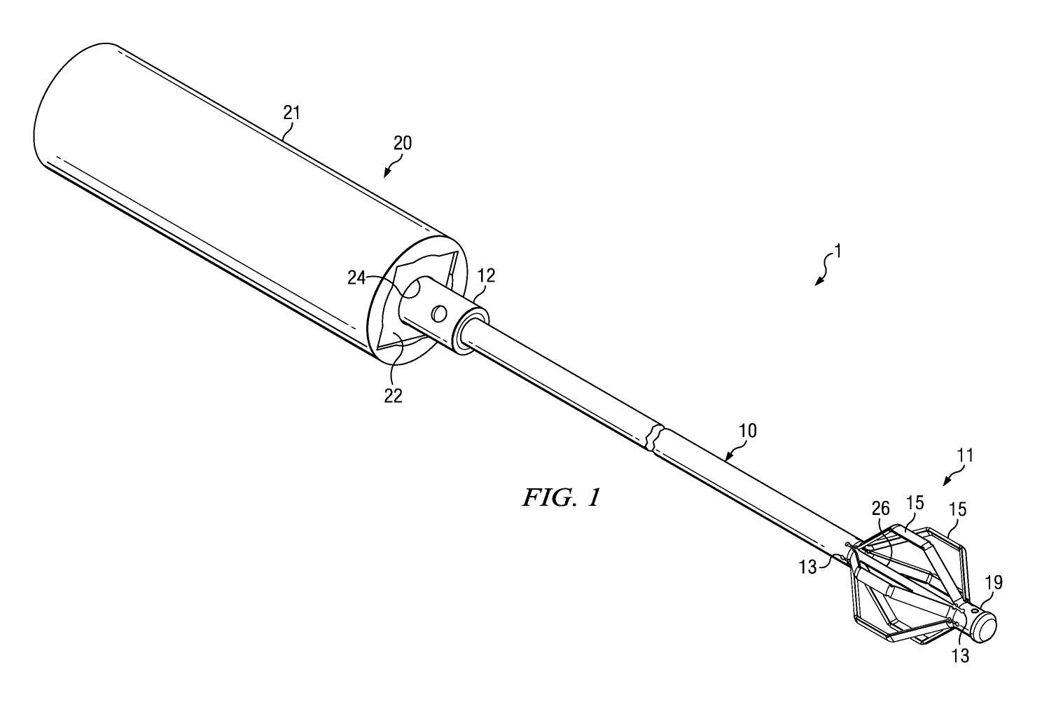

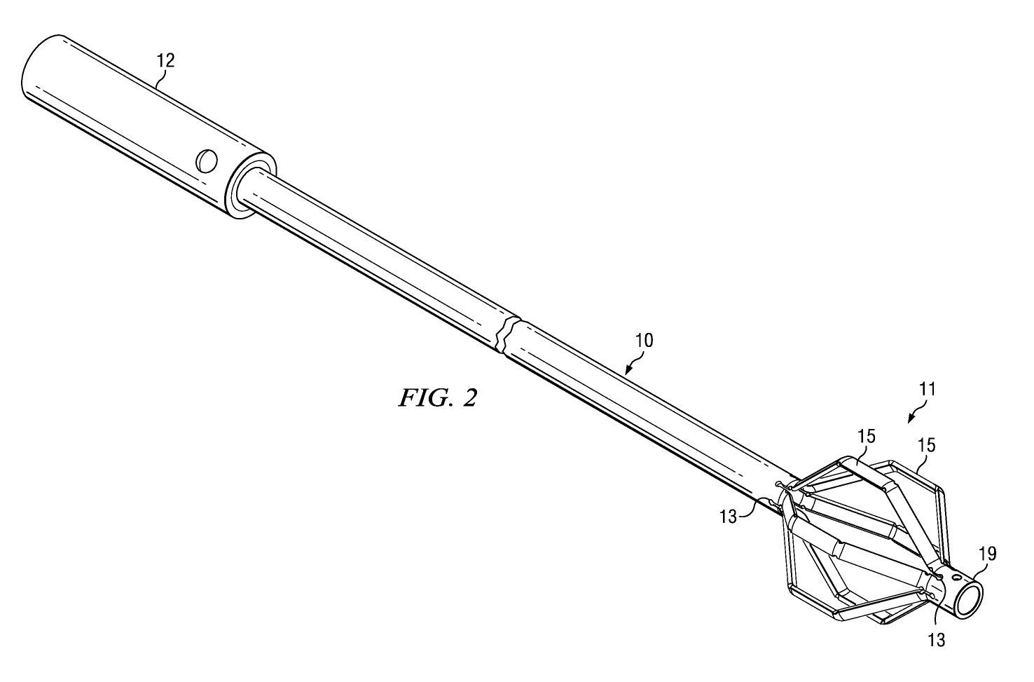

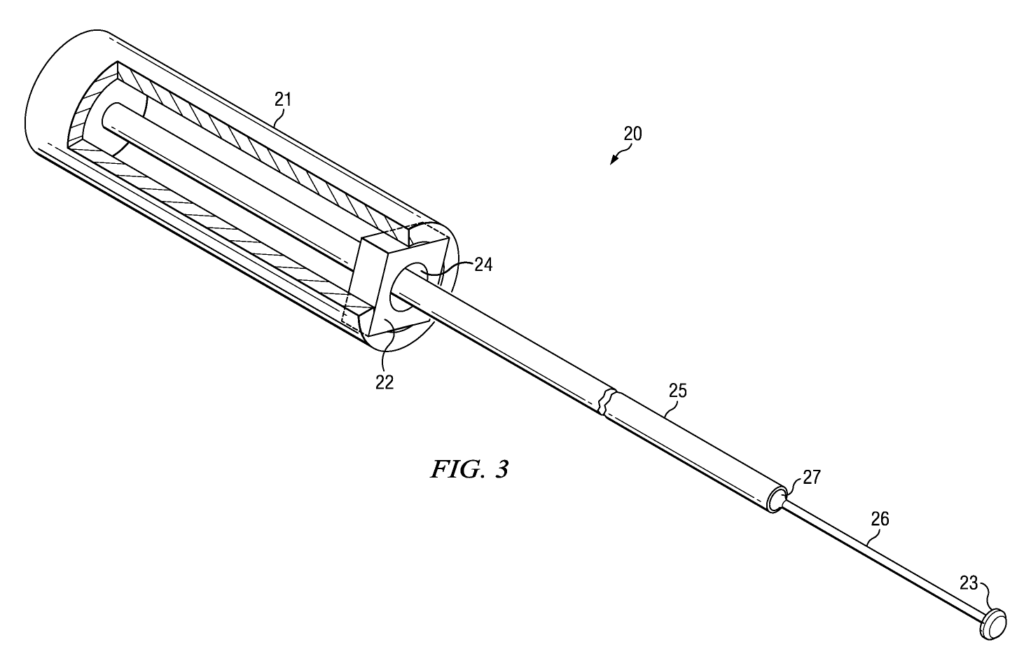

A surgical cutting tool 1 that includes a tubular shaft 10 with an expandable blade 11 comprising one or more cuts 13 in the tubular shaft 10 is shown in FIG. 1. Various embodiments of a tubular shaft may not be tubular along their entire length or even a significant portion of their length. For example and without limitation, a tubular shaft embodiment may only have outer wall or tubular portions where an expandable blade is formed. FIG. 1 depicts an assembled surgical cutting tool 1, and FIGS. 2 and 3 respectively show portions of the surgical cutting tool 1 of FIG. 1, with complementary portions of the surgical cutting tool 1 removed to more clearly illustrate the surgical cutting tool 1. The surgical cutting tool 1 shown includes an actuator member 20. The illustrated expandable blade 11 is near a distal end 19 of the tubular shaft 10. The expandable blade 11 is shown deployed substantially radially from the tubular shaft 10. In some embodiments, radially extending elements 15 may be extendable from the tubular shaft 10 parallel with a longitudinal axis of the tubular shaft 10. In other embodiments, radially extending elements may be extendable from the tubular shaft 10 along a helical or other non-parallel path with a longitudinal axis of the tubular shaft 10.

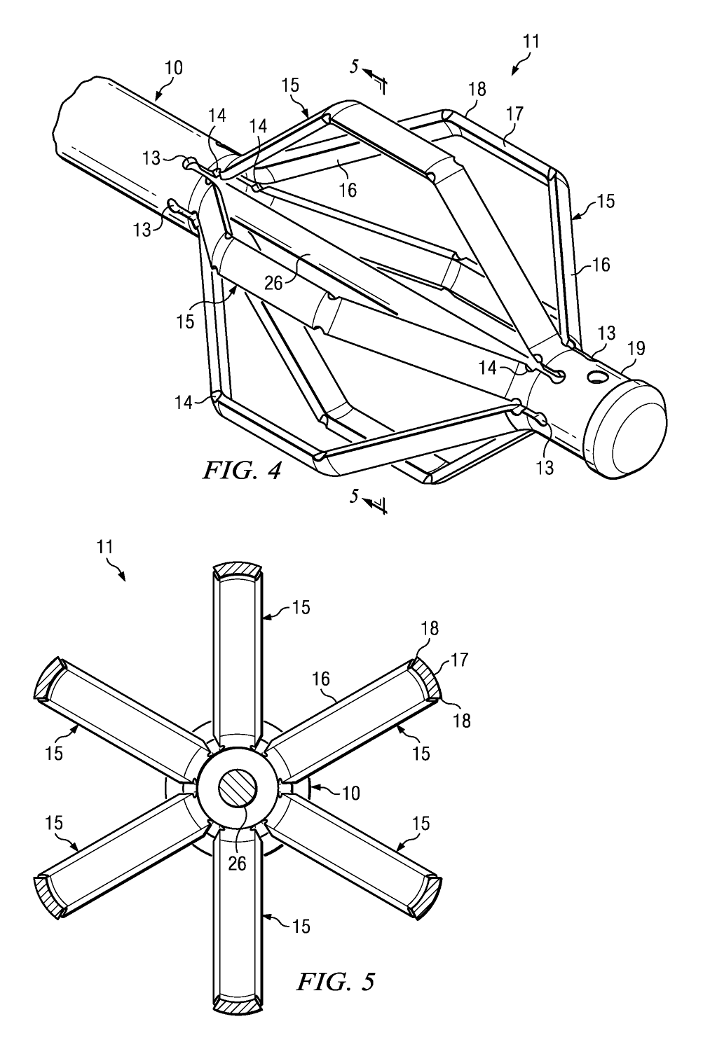

Referring to FIG. 4, the tubular shaft 10 includes multiple cuts 13 to at least in part form the expandable blade 11. The cuts 13 may be any type of opening or partial opening and do not necessarily require an act of cutting in their formation. The cuts 13 may be milled, cut, etched, burned, abraded, cast, or formed in the tubular shaft 10 in any effective way. The illustrated cuts 13 include stress relieved areas 14 designed to one or more of encourage bending at particular points and prevent areas of increased stress where ruptures in the material of the expandable blade 11 may be more likely to occur during use of the expandable blade 11. The expandable blade 11 is shown in FIGS. 4 and 5 with six substantially radially extending elements 15 deployed from the tubular shaft 10. As illustrated, the substantially radially extending elements 15 deploy in six substantially opposite directions. That is, each element of three pairs of substantially radially extending elements 15 are opposite from one another. In other embodiments, there may be two, four, eight, or any effective number of opposite substantially radially extending elements. In some embodiments, there may only be one or any odd number of substantially radially extending elements. In some embodiments, the substantially radially extending elements may not be opposite from one another. A relatively thinner portion 26 of a rod 25 as described below is shown engaged with the tubular shaft 10 in FIGS. 1, 3, 4 and 5.

Each substantially radially extending element 15 may include two substantially opposite end portions 16 that couple with a main body of the tubular shaft 10 and a central portion 17 between the two substantially opposite end portions 16, as shown in FIGS. 4 and 5. In other embodiments, each substantially radially extending element 15 may be any effective size or number of components. The central portion 17 may include elements for cutting tissue. For example, one or more edges of the central portion 17 may be sharpened, serrated, or otherwise configured to cut or engage tissue. Edges 18 of the illustrated embodiment have been formed at a sharp angle to enable the cutting of tissue. In other embodiments, cutting edges or other elements for cutting may be incorporated along any portion of an expandable blade. For example and without limitation, cutting edges may be incorporated into one or both of the substantially opposite end portions 16. The substantially opposite end portions 16, the central portion 17, and the tubular shaft 10 may be formed from a single material or may include a number of materials joined together in any effective way. Similarly, any substantially radially extending element may be made from a common material with a tubular shaft from which it extends, or may be a hybrid of more than one material. For example and without limitation, a portion of a substantially radially extending element designed for cutting may be made from a material more capable of effectively sustaining a sharp edge and another portion that will be required to bend during operation of the device may be made from a softer, more flexible material.

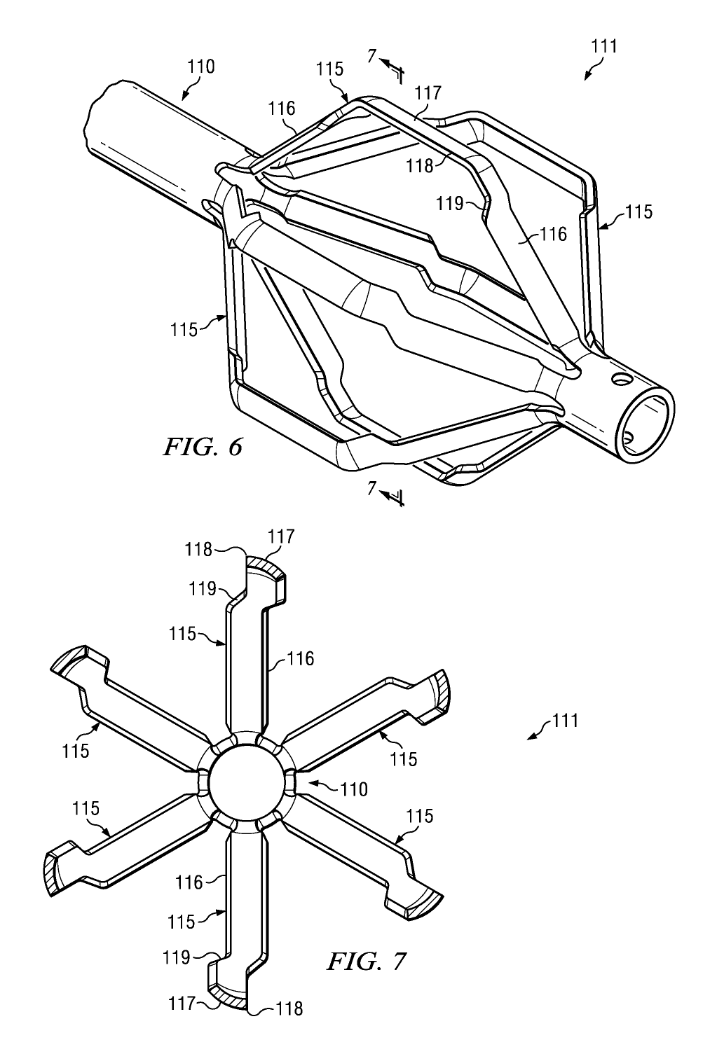

Embodiments of expandable blades 111, 211 are illustrated in FIGS. 6-10. The expandable blades 111, 211 are similar to the expandable blade 11 in several ways; however, the expandable blades 111, 211 also include central portions 117, 217 that are offset from at least portions of their respective opposite ends 116, 216. The term offset as used herein with regard to some embodiments means offset from a direct radial extension.

FIGS. 6-8 illustrate the expandable blade 111 with substantially radially extending elements 115 that include two substantially opposite end portions 116 that couple with a main body of a tubular shaft 110. The expandable blade 111 may be formed from the tubular shaft 110 by making multiple cuts or in or manipulation of the expandable blade 111, as similarly noted above with regard to the expandable blade 11. A central portion 117 is shown between the two substantially opposite end portions 116. In other embodiments, each substantially radially extending element 115 may be any effective size or number of components. The central portion 117 may include elements for cutting tissue. For example, one or more edges of the central portion 117 may be sharpened, serrated, or otherwise configured to cut or engage tissue. Edges 118 of the illustrated embodiment have been formed at a sharp angle to enable the cutting of tissue. In other embodiments, cutting edges or other elements for cutting may be incorporated along any portion of an expandable blade. For example and without limitation, cutting edges may be incorporated into one or both of the substantially opposite end portions 116. The substantially opposite end portions 116, the central portion 117, and the tubular shaft 110 may be formed from a single material or may include a number of materials joined together in any effective way. Similarly, any substantially radially extending element 115 may be made from a common material with the tubular shaft 110 from which it extends, or may be a hybrid of more than one material. For example and without limitation, a portion of a substantially radially extending element 115 designed for cutting may be made from a material more capable of effectively sustaining a sharp edge, and another portion that will be required to bend during operation of the device may be made from a softer, more flexible material.

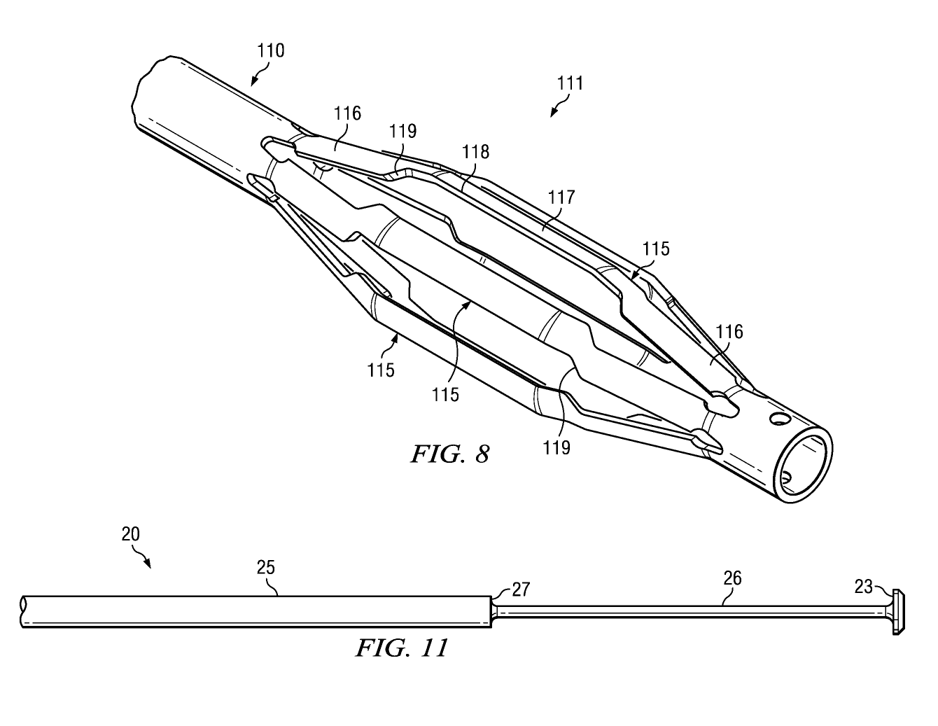

The expandable blade 111 is shown in an expanded state in FIG. 6 and in a contracted state in FIG. 8. In both FIG. 6 and FIG. 8, a pattern of cuts is illustrated in the tubular shaft 110 that causes the central portion 117 to be offset from the substantially opposite end portions 116 at an elbow 119. The elbow 119 and consequent offset, as shown in FIG. 7, assists in projecting the edge 118 away from parts of the substantially radially extending element 115 to better provide a tool that effectively cuts and detaches material against which the expandable blade 111 is passed in an expanded state. As shown in FIG. 8, when the expandable blade 111 is in a contracted state, the edge 118 is drawn inwardly and does not project from the general perimeter of the expandable blade 111. Therefore, the expandable blade 111 in a contracted state may be passed by tissue without danger of the edge 118 cutting the tissue.

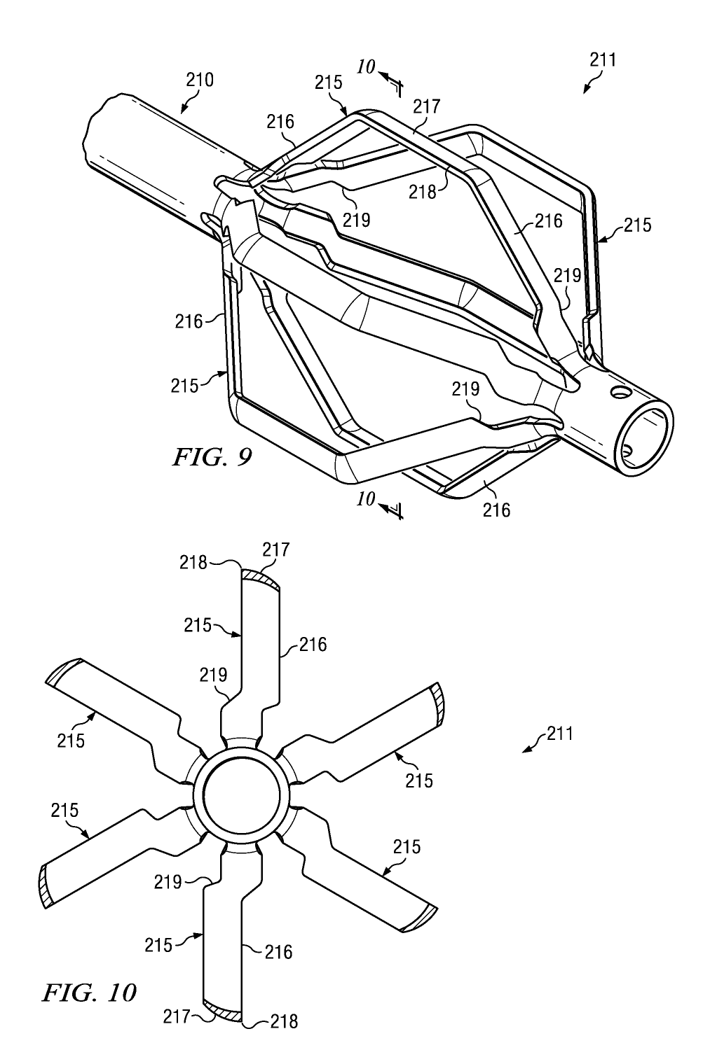

FIGS. 9 and 10 illustrate the expandable blade 211 with substantially radially extending elements 215 that include two substantially opposite end portions 216 that couple with a main body of a tubular shaft 210 and a central portion 217 between the two substantially opposite end portions 216. In other embodiments, each substantially radially extending element 215 may be any effective size or number of components. The expandable blade 211 may be formed from the tubular shaft 210 by making multiple cuts or in or manipulation of the expandable blade 211, as similarly noted above with regard to the expandable blade 11. The central portion 217 may include elements for cutting tissue. For example, one or more edges of the central portion 217 may be sharpened, serrated, or otherwise configured to cut or engage tissue. Edges 218 of the illustrated embodiment have been formed at a sharp angle to enable the cutting of tissue. In other embodiments, cutting edges or other elements for cutting may be incorporated along any portion of an expandable blade. For example and without limitation, cutting edges may be incorporated into one or both of the substantially opposite end portions 216. The substantially opposite end portions 216, the central portion 217, and the tubular shaft 210 may be formed from a single material or may include a number of materials joined together in any effective way. Similarly, any substantially radially extending element 215 may be made from a common material with the tubular shaft 210 from which it extends, or may be a hybrid of more than one material. For example and without limitation, a portion of any of a substantially radially extending elements 215 designed for cutting may be made from a material more capable of effectively sustaining a sharp edge, and another portion that will be required to bend during operation of the device may be made from a softer, more flexible material.

The expandable blade 211 is shown in an expanded state in FIG. 9. A pattern of cuts is illustrated in the tubular shaft 210 that causes the central portion 217 to be offset from at least portions of the substantially opposite end portions 216 at an elbow 219. Respective elbows 219 are located at intermediate points within each of the substantially opposite end portions 216. This elbow and offset location is in contrast to the elbows 119, but provides similar offset characteristics. Particularly, the elbow 219 and consequent offset, as shown in FIG. 10, assists in projecting the edge 218 away from parts of the substantially radially extending element 215 to better provide a tool that effectively cuts and detaches material against which the expandable blade 211 is passed in an expanded state. When the expandable blade 211 is in a contracted state, the edge 218 is drawn inwardly and does not project from the general perimeter of the expandable blade 211. Therefore, the expandable blade 211 in a contracted state may be passed by tissue without danger of the edge 218 cutting the tissue.

The surgical cutting tool 1 illustrated in FIGS. 1-3 shows the actuator member 20 extending beyond the distal end 19 of the tubular shaft 10 (FIG. 1). The actuator member 20 includes a bearing surface 23, as better illustrated in FIG. 11 and as referenced in FIG. 3. The actuator member 20 shown includes a handle 21 near its proximal end. The handle 21 is coupled with a collar 22 near the distal end 19 of the handle 21. The collar 22 includes an opening 24 through which the rod 25 that is coupled with the handle 21 at the proximal end of the rod 25 may pass. The rod 25 is disposed through the tubular shaft 10, as illustrated in FIG. 1, and the tubular shaft 10 also passes through the opening 24 in the collar 22. The actuator member 20 may be disposed through the middle of the tubular shaft 10, as illustrated in FIG. 1. Alternatively or in addition, some or all of an actuator member of some embodiments may be through any portion of the area within a tubular shaft, or may be disposed adjacent to a tubular shaft or an at least partially non-tubular shaft. The rod 25 shown in FIGS. 3 and 11 includes the relatively thinner portion 26 with a distal face 27 at the transition between the relatively thinner portion 26 and the more proximal end of the rod 25, which is relatively thicker.

The illustrated bearing surface 23 is configured to press against the distal end 19 of the tubular shaft 10 when the actuator member 20 is moved relative to the tubular shaft 10 in a first direction to deploy the expandable blade 11. As shown in FIG. 1, the actuator member 20 has been moved relative to the tubular shaft 10 in the first direction to deploy the expandable blade 11. Relative movement between the actuator member 20 and the tubular shaft 10 may be translational movement, rotational movement to generate translational movement, or by any other effective mechanism to generate relative movement. For example and without limitation, the proximal end of the tubular shaft 10 includes a flange 12 that may include threads on its outer surface that interact with internal threads that may be present on some embodiments of the collar 22 of the actuator member 20. Therefore, by rotating the actuator member 20 relative to the tubular shaft 10, for this embodiment, translational movement is generated between the actuator member 20 and the tubular shaft 10. Alternatively, the flange 12 may slide or translate directly within the collar 22. Some embodiments may include locking, ratcheting, gears, or other mechanisms to control or induce motion between the actuator member 20 and the tubular shaft 10 or components of either relative to the other.

In the embodiment illustrated in FIGS. 1-3, the bearing surface 23 (FIGS. 3 and 11) of the actuator member 20 is not connected to the tubular shaft 10 such that substantially any tensile resistance exists between the tubular shaft 10 and the actuator member 20. That is, when the actuator member 20 is moved relative to the tubular shaft 10 in a second direction that is substantially opposite from the first direction of movement that deploys the expandable blade 11, there is substantially no tensile resistance between the actuator member 20 and the tubular shaft 10. In some embodiments, insubstantial tensile resistance such as adhesion between the tubular shaft 10 and the bearing surface 23 or an insubstantial tensile connection in place to facilitate or as a result of handling, shipping, or cleaning may exist, but would not be considered connected as specified herein. Actuator members of other embodiments are configured to apply force to a tubular shaft by any effective mechanism and are not limited to application of force through a device such as the bearing surface 23.

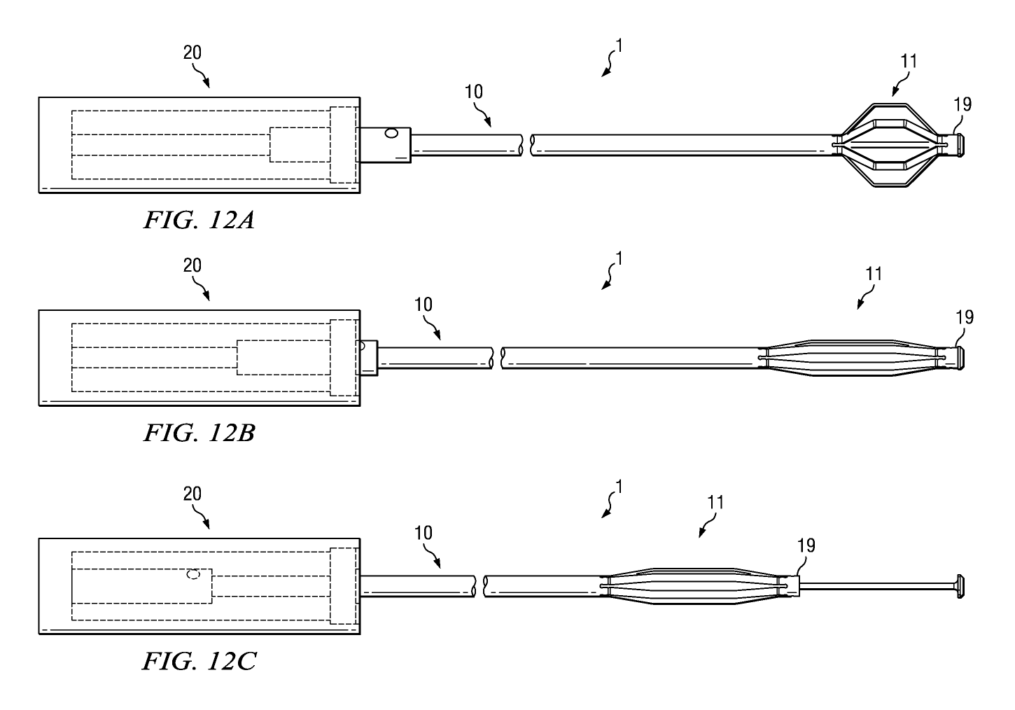

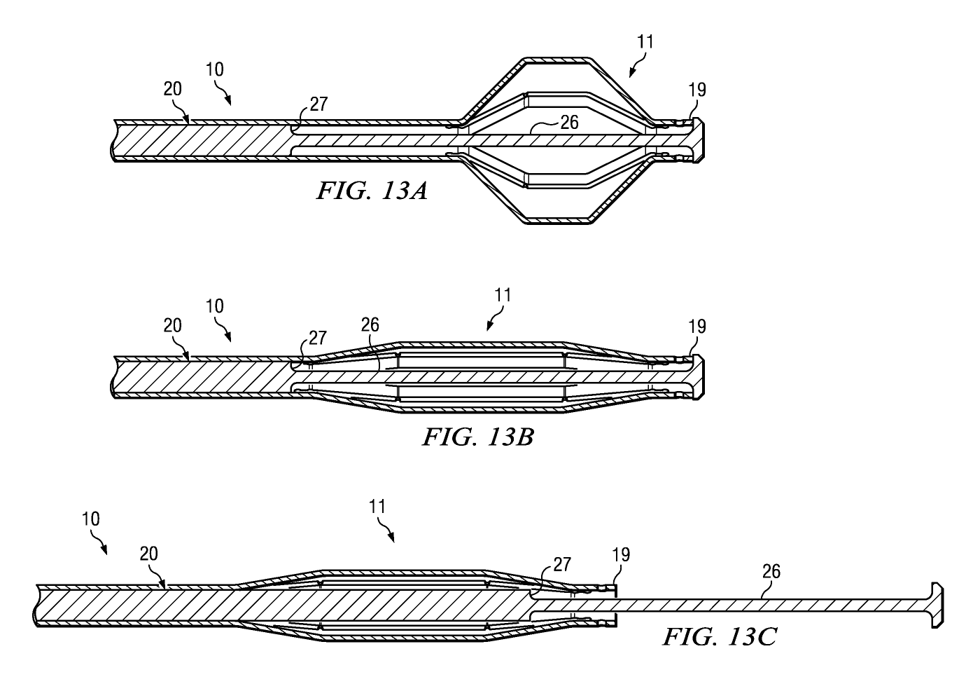

An actuator member of some embodiments may also include or serve as a plunger configured to push material through an expandable blade and out of a distal end of a tubular shaft. For example, as illustrated in FIGS. 12A-C and 13A-C, the actuator member 20 is configured to plunge, or push, material that has been removed from a patient and captured within the expandable blade 11 through the expandable blade 11 and out of the distal end 19 of the tubular shaft 10. As shown in a first state in FIGS. 12A and 13A with the actuator member 20 moved in a first direction relative to the tubular shaft 10, the expandable blade 11 is expanded and in a cutting position. In use, the expandable blade 11 may be used to detach material such as tissue that is subsequently captured within extents of the expandable blade 11. The actuator member 20 is shown in FIGS. 12B and 13B moved partially in a second direction relative to the tubular shaft 10 that is substantially opposite from the first direction. In this position, the expandable blade 11 is contracted around the relatively thinner portion 26 (FIG. 13B) of the actuator member 20, and some or all of the material may be captured between the expandable blade 11 and the relatively thinner portion 26. The distal face 27 is shown in FIG. 13B near a proximal portion of the expandable blade 11. The actuator member 20 is shown in FIGS. 12C and 13C moved further in the second direction. Between this position and the position illustrated in FIGS. 12B and 13B, the distal face 27 may push material through the expandable blade 11 and out of the distal end 19 of the tubular shaft 10. This action may be useful in some embodiments to clear out the expandable blade 11 so that it may be effectively redeployed to a surgical site. In some embodiments, the surgical cutting tool is removed from the surgical site after the state illustrated in FIGS. 12B and 13B is achieved, and the state illustrated in FIGS. 12C and 13C is reached while the surgical cutting tool is away from a surgical site, and in some instances, outside of a patient's body. In addition or alternatively, the surgical cutting tool 1 may be used in this manner to collect tissue for the purpose of conducting a biopsy.

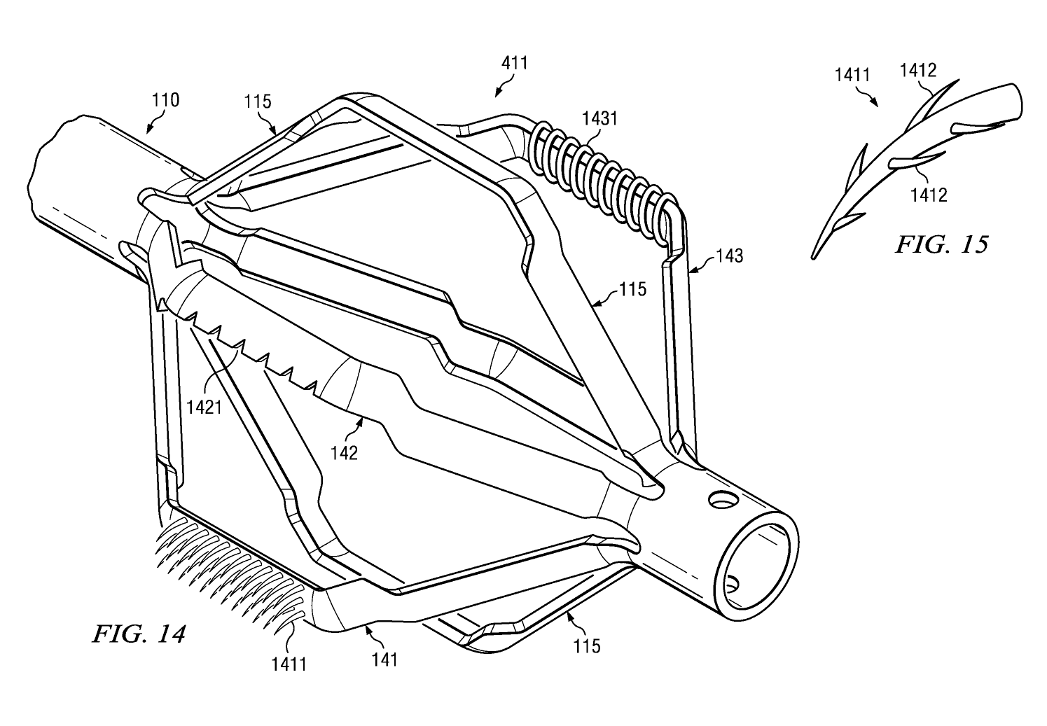

Embodiments of an expandable blade may also include one or more of bristles, barbs, reentrant cuts, woven, close-proximity and wound strands, and sharpened edges configured to detach, cut, capture, or otherwise manipulate material, such as but not limited to, tissue. An example expandable blade 411 is illustrated in FIG. 14. The expandable blade 411 includes multiple substantially radially extending elements 115, 141, 142, 143 with various features. In some embodiments, each of the substantially radially extending elements may include any one or more of the features of the illustrated substantially radially extending elements 115, 141, 142, 143. Substantially radially extending element 141 includes attached bristles 1411. One embodiment of a bristle is shown in FIG. 15. A single bristle 1411 is illustrated in FIG. 15 with barbs 1412 that may increase the ability of the bristles 1411 to effectively engage tissue. The barbs 1412 are illustrated pointed toward a base of the bristle 1411. In other embodiments, one or more barbs 1412 may be pointed toward a free end of a bristle. In some embodiments, bristles may not include barbs. Bristles may be one or more of relatively smooth, made from a course material that creates friction with tissue, or may be very stiff such that each bristle tends to cut into or abrade tissue over which the bristle is passed. The bristles 1411 may be arrange on any portion of the substantially radially extending element 141 and in any configuration. For example, bristles 1411 may be placed on a central portion of the radially extending element 141 as shown. Bristles may be alternatively or in addition located on one or both of opposite end portions of the radially extending element 141 that couple with the main body of the tubular shaft 110. Bristles 1411 may be on the outside of the substantially radially extending element 141, as illustrated, and may be on the inside of the substantially radially extending element 141. Bristles that are sufficiently stiff and close together may also capture or pinch material between the bristles. The bristles 1411 or other embodiments of bristles may be arranged on a substantially radially extending element 141 in rows as illustrated, or may be place randomly or in any effective pattern. Bristles may be generally straight, as illustrated, or may be curved, helical, bent at particular angles, or of any other effective configuration.

Substantially radially extending element 142 includes reentrant cuts 1421 that form a serrated edge along the substantially radially extending element 142. The reentrant cuts 1421 may act in combination with a sharpened edge into which they are cut, or may be cut into a blunt edge and be the sole mechanism for capturing or cutting tissue along a substantially radially extending element. Reentrant cuts 1421 may be formed in a central portion of the substantially radially extending element 142 as shown. Reentrant cuts 1421 may be alternatively or in addition located on one or both of opposite end portions of the substantially radially extending element 142 that couple with the main body of the tubular shaft. Reentrant cuts 1421 may be located on either or both of front and back edges of the substantially radially extending elements 142.

Substantially radially extending element 143 includes a strand 1431 wrapped around a portion of the substantially radially extending element 143. The strand 1431 may act in combination with one or more sharpened edges around which the strand 1431 is wrapped, or may be wrapped around a blunt edge and may be the sole mechanism for engaging tissue along a substantially radially extending element. The strand 1431 may be one or more of relatively smooth, made from a course material that creates friction with tissue, or may be very stiff and include sharp portions that tend to cut into or abrade tissue over which the strand 1431 is passed. In some embodiments, the strand 1431 is configured to capture material between respective portions of the strand 1431 or between the strand 1431 and the substantially radially extending element 143. The strand 1431 may be located around a central portion of the substantially radially extending element 143 as shown, or may alternatively or in addition be located on one or both of opposite end portions of the substantially radially extending element 143 that couple with the main body of the tubular shaft 110. In addition or alternatively, the strand 1431 may be replaced with a woven or randomly oriented fiber material that includes similar characteristics to the characteristics of the strand 1431. For example, one embodiment may include a material with randomly oriented fibers similar to “steel wool.”

In some embodiments, all or a portion of an expandable blade may be implanted at a surgical site to support treated tissues. Alternatively, embodiments of the invention may also include a medical implant to be delivered to a surgical site along the same path as the surgical cutting tool and then expanded to treat tissues at the surgical site.

In some embodiments, the outside diameter of the distal end of the surgical cutting tool, including all expandable components in their contracted state is equal to or less than 6 mm. An outside diameter of 6 mm or less may be achieved by one or both of releasing the surgical cutting tool to a contracted state or by moving the surgical cutting tool through an opening of 6 mm or less. In some embodiments, the outside diameter of the distal end of the surgical cutting tool, including all expandable components in their contracted state is between 8 mm and 12 mm.

A fill material may be introduced at a surgical site in combination with a medical implant and/or following use of a surgical cutting tool as disclosed herein. The fill material may be a paste, gel, liquid, suspension, granular mixture, or similar substance. Non-limiting examples of fill materials include bone paste, morselized allograft, autograft, or xenograft bone, ceramics, or various polymers. The fill material may be a material that hardens after implantation. Some fill materials which are not necessarily hardenable or curable may be used in association with the present invention. For example, the fill material may comprise beads or small particles or grains of material, some of which may, in aggregate, achieve a harder consistency as a result of interlocking or compaction. In some embodiments, the fill material may also include a bone growth promoting substance. Osteogenic or bone growth promoting substances may include, without limitation, autograft, allograft, xenograft, demineralized bone, synthetic and natural bone graft substitutes, such as bioceramics and polymers, and osteoinductive factors. A separate carrier to hold materials within the device may also be used. These carriers may include collagen-based carriers, bioceramic materials, such as BIOGLASS®, hydroxyapatite and calcium phosphate compositions. The carrier material may be provided in the form of a sponge, a block, folded sheet, putty, paste, graft material or other suitable form. The osteogenic compositions may include an effective amount of a bone morphogenetic protein (BMP), transforming growth factor β1, insulin-like growth factor, platelet-derived growth factor, fibroblast growth factor, LIM mineralization protein (LMP), and combinations thereof or other therapeutic or infection resistant agents, separately or held within a suitable carrier material. Introduction of fluid or fill material into an expandable medical implant embodiment may be through a syringe or similar device, through direct placement, or by any other effective mechanism.

Embodiments of the devices in whole or in part may be constructed of biocompatible materials of various types. Examples of materials include, but are not limited to, shape-memory alloys, titanium, titanium alloys, cobalt chrome alloys, stainless steel, non-reinforced polymers, carbon-reinforced polymer composites, PEEK and PEEK composites, low density polyethylene, ceramics and combinations thereof.

The surgical cutting tool presents a small profile in its contracted state so that it is well-suited for use from any surgical approach. Embodiments of the invention may include implantation from any surgical approach, including but not limited to anterior, posterior, lateral, oblique, or by any combination of these and other approaches.

An embodiment of the invention is a method of implanting an expandable medical implant. Some method embodiments include providing a surgical cutting tool configured to change between a contracted state wherein the diameter of the distal end and working components of the surgical cutting tool are less than or equal to about 8 mm and an expanded state wherein the diameter of the working components of the surgical cutting tool are expanded to prepare a disc space. Method embodiments may further include inserting the surgical cutting tool between vertebral endplates in a contracted state, expanding the surgical cutting tool, and manipulating the surgical cutting tool to remove and capture tissue within the surgical cutting tool. Method embodiments may further include returning the surgical cutting tool to a contracted state, removing the surgical cutting tool from between vertebral endplates, and operating the surgical cutting tool to expel tissue captured in the surgical cutting tool from the surgical cutting tool. Method embodiments may include inserting an expandable medical implant between the vertebral endplates in a contracted state and deploying the expandable medical implant. Some embodiments include the act of inserting an expandable medical implant with a diameter less than or equal to about 8 mm between the vertebral endplates and expanding the expandable medical implant to provide support between the vertebral endplates. Some embodiments include the act of leaving at least a portion of the surgical cutting tool between the vertebral endplates to provide support between the vertebral endplates. Some embodiments include placing a fill material between the vertebral endplates. Some embodiments include implanting supplemental fixation devices to stabilize vertebrae.

Various method embodiments of the invention are described herein with reference to particular surgical tools and implants. However, in some circumstances, each disclosed method embodiment may be applicable to each of the surgical tools and implants, or to some other implant operable as disclosed with regard to the various method embodiments.

Terms such as proximal, distal, anterior, posterior, lateral, and the like have been used herein to note relative positions. However, such terms are not limited to specific coordinate orientations, but are used to describe relative positions referencing particular embodiments. Such terms are not generally limiting to the scope of the claims made herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. A surgical cutting tool comprising:
a tubular shaft with an expandable blade comprising one or more cuts in the tubular shaft, wherein the expandable blade is near a distal end of the tubular shaft, and wherein the expandable blade deploys substantially radially from the tubular shaft; and
an actuator member that extends beyond the distal end of the tubular shaft and includes a bearing surface configured to press against the distal end of the tubular shaft when the actuator member is moved relative to the tubular shaft in a first direction to deploy the expandable blade;
wherein the bearing surface is not connected to the tubular shaft such that substantially any tensile resistance exists between the tubular shaft and the actuator member when the actuator member is moved relative to the tubular shaft in a second direction that is substantially opposite from the first direction.

2. The surgical cutting tool of claim 1 wherein the expandable blade deploys substantially radially in at least two substantially opposite directions.

3. The surgical cutting tool of claim 1 wherein the expandable blade includes bristles configured to capture tissue.

4. The surgical cutting tool of claim 1 wherein the expandable blade includes cutting edges.

5. The surgical cutting tool of claim 1 wherein the expandable blade includes one or more substantially radially extending elements, each substantially radially extending element comprising:
two substantially opposite end portions that couple with a main body of the tubular shaft, and
a central portion between the two substantially opposite end portions that includes elements for cutting tissue,
wherein the central portion is offset from at least portions of the two substantially opposite ends.

6. The surgical cutting tool of claim 1 wherein the actuator member is disposed through the middle of the tubular shaft.

7. The surgical cutting tool of claim 1 wherein the actuator member includes a plunger configured to push material through the expandable blade and out of the distal end of the tubular shaft.

8. The surgical cutting tool of claim 7 wherein the actuator member includes a relatively thinner portion nearer the distal end of the actuator member and a relatively thicker portion proximal of the relatively thinner portion wherein a distal face of the thicker portion is configured to push material through the expandable blade.

9. A surgical cutting tool comprising:
a tubular shaft with an expandable blade comprising one or more cuts in the tubular shaft, wherein the expandable blade is near a distal end of the tubular shaft, and wherein the expandable blade deploys substantially radially from the tubular shaft with one or more substantially radially extending elements, each substantially radially extending element comprising:
two substantially opposite end portions that couple with a main body of the tubular shaft, and
a central portion between the two substantially opposite end portions that includes elements for cutting tissue,
wherein the central portion is offset from at least portions of the two substantially opposite ends; and
an actuator member configured to apply force to the tubular shaft when the actuator member is moved relative to the tubular shaft in a first direction to deploy the expandable blade.

10. The surgical cutting tool of claim 9 wherein the expandable blade deploys substantially radially in at least two substantially opposite directions.

11. The surgical cutting tool of claim 9 wherein the expandable blade includes bristles configured to capture tissue.

12. The surgical cutting tool of claim 9 wherein the expandable blade includes cutting edges.

13. The surgical cutting tool of claim 9 wherein the actuator member extends beyond the distal end of the tubular shaft and includes a bearing surface configured to apply force to the distal end of the tubular shaft when the actuator member is moved relative to the tubular shaft in the first direction to deploy the expandable blade, and wherein the bearing surface is not connected to the tubular shaft such that substantially any tensile resistance exists between the tubular shaft and the actuator member when the actuator member is moved relative to the tubular shaft in a second direction that is substantially opposite from the first direction.

14. The surgical cutting tool of claim 9 wherein the actuator member includes a plunger configured to push material through the expandable blade and out of the distal end of the tubular shaft.

15. The surgical cutting tool of claim 14 wherein the actuator member includes a relatively thinner portion nearer the distal end of the actuator member and a relatively thicker portion proximal of the relatively thinner portion wherein a distal face of the thicker portion is configured to push material through the expandable blade.

16. A surgical cutting tool comprising:
a tubular shaft with an expandable blade comprising one or more cuts in the tubular shaft, wherein the expandable blade is near a distal end of the tubular shaft, and wherein the expandable blade deploys substantially radially from the tubular shaft; and
an actuator member that extends beyond the distal end of the tubular shaft and includes a bearing surface configured to press against the distal end of the tubular shaft when the actuator member is moved relative to the tubular shaft in a first direction to deploy the expandable blade;
wherein the actuator member includes a plunger configured to push material through the expandable blade and out of the distal end of the tubular shaft.

17. The surgical cutting tool of claim 16 wherein the expandable blade deploys substantially radially in at least two substantially opposite directions.

18. The surgical cutting tool of claim 16 wherein the expandable blade includes bristles configured to capture tissue.

19. The surgical cutting tool of claim 16 wherein the expandable blade includes cutting edges.

20. The surgical cutting tool of claim 16 wherein the expandable blade includes one or more substantially radially extending elements, each substantially radially extending element comprising:
two substantially opposite end portions that couple with a main body of the tubular shaft, and
a central portion between the two substantially opposite end portions that includes elements for cutting tissue,
wherein the central portion is offset from at least portions of the two substantially opposite ends.

21. The surgical cutting tool of claim 16 wherein the bearing surface is not connected to the tubular shaft such that substantially any tensile resistance exists between the tubular shaft and the actuator member when the actuator member is moved relative to the tubular shaft in a second direction that is substantially opposite from the first direction.

22. The surgical cutting tool of claim 16 wherein the actuator member includes a relatively thinner portion nearer the distal end of the actuator member and a relatively thicker portion proximal of the relatively thinner portion wherein a distal face of the thicker portion is configured to push material through the expandable blade.
